(19) United States
(12) Patent Application Publication
(10) Pub. No.: US 2026/0103724 A1
(43) Pub. Date: Apr. 16, 2026

(54) VECTOR FOR MULTI-GENE BASE EDITING, EDITING METHOD, AND USE

(71) Applicant: Agricultural Genomics Institute at Shenzhen,Chinese Academy of Agricultural Sciences, Shenzhen, Guangdong (CN)

(72) Inventors: Kui LI, Shenzhen, Guangdong (CN); Lei HUANG, Shenzhen, Guangdong (CN); Jinxue RUAN, Shenzhen, Guangdong (CN); Bo LI, Shenzhen, Guangdong (CN)

(21) Appl. No.: 19/013,137

(22) Filed: Jan. 8, 2025

(30) Foreign Application Priority Data
Oct. 11, 2024 (CN) 202411415041.7

(51) Int. Cl. C12N 15/85; C07K 14/005; C12N 9/22; C12N 15/11; C12N 15/64

(52) U.S. Cl. CPC C12N 15/85; C07K 14/005; C12N 9/22; C12N 15/111; C12N 15/64; C12N 2310/20; C12N 2710/16222; C12N 2820/60

(57) ABSTRACT

Provided are a vector for multi-gene base editing, an editing method, and a use, and belongs to the technical field of gene editing. This application provides a vector for multi-gene base editing. In the vector, a replicon element, U6, and sgRNA are linked in series to allow a plasmid carrying the replicon element to persist in a cell, such that other elements on the plasmid can be continuously expressed in the cell, which improves the efficiency of multi-gene silencing. This application also provides a tandem vector, which is obtained by removing an epi element from the vector above. The two multi-gene editing vectors constructed in the embodiments of the present disclosure both have a silencing editing effect for target loci. The vector including the epi element has a higher editing efficiency than the vector without the epi element.

### CROSS REFERENCE TO RELATED APPLICATION

This patent application claims the benefit and priority of Chinese Patent Application No. 2024114150417, filed with the China National Intellectual Property Administration on Oct. 11, 2024, the disclosure of which is incorporated by reference herein in its entirety as part of the present application.

### REFERENCE TO SEQUENCE LISTING

A computer readable XML file entitled “GWP20241007167”, that was created on Dec. 20, 2024, with a file size of about 104,343 bytes, contains the sequence listing for this application, has been filed with this application, and is hereby incorporated by reference in its entirety.

### TECHNICAL FIELD

The present disclosure belongs to the technical field of gene editing, and specifically relates to a vector for multi-gene base editing, an editing method, and a use.

### BACKGROUND

The gene knockout is a gene editing technique that destroys a normal sequence of a target gene to cause the loss of a normal function of the target gene. Since the advent of the CRISPR/Cas9 system, the operation of gene knockout has been increasingly simple, and the cost of gene knockout has gradually decreased. Therefore, the application of gene knockout systems in the biomedical field has gradually increased. In the field of animal breeding, the gene knockout technique also has a broad application prospect. Studies have shown that the knockout of specific genes can increase the disease resistance of pigs and improve the economic benefits. Since a superior trait is often controlled by multiple genes, the knockout of multiple genes is of great significance for breeding. To edit multiple genes simultaneously, the editing efficiency needs to be further improved, especially for a homozygous cell line requiring a multi-gene knockout. Therefore, the improvement of the multi-gene editing efficiency is an important link for breeding and even agricultural development. The current gene knockout method is mainly as follows: a target gene in cells is cleaved with the CRISPR/Cas9 system to produce a double-strand break, and then a cleaved gene is repaired with a repair system of the cells themselves to make a repaired gene have a base insertion or deletion, resulting in a frameshift mutation to cause the loss of a function of the gene. However, the double-strand break will cause an adverse impact on the cells, and may activate proto-oncogenes and inhibit tumor suppressor genes.

### SUMMARY

The present disclosure provides a vector for multi-gene base editing, an editing method, and a use. In the present disclosure, through a cytosine base editing system, bases on exons are mutated to produce premature termination codons to cause multi-gene silencing, such that target genes cannot be expressed normally and harms such as double-strand breaks can be avoided.

The present disclosure provides a vector for multi-gene base editing, including a replicon element, a U6 promoter, and sgRNA for each target gene that are linked in series, where each U6 promoter initiates expression of a sgRNA sequence of a target gene.

In a specific embodiment of the present disclosure, the replicon element includes EBNA1 and OriP.

In a specific embodiment of the present disclosure, there is no less than 1 target gene.

In a specific embodiment of the present disclosure, the target gene includes a gene related to a pig breeding field and a gene related to a pork quality.

In a specific embodiment of the present disclosure, a backbone vector for the vector includes the following elements: a single-base editor, an epi replicon element, and a plurality of U6 promoters linked in series.

The present disclosure also provides a tandem vector for multi-gene base editing, where a preparation method of the tandem vector includes: removing the replicon element from the vector described above.

In a specific embodiment of the present disclosure, the preparation method of the tandem vector includes: subjecting the vector described above to double enzyme digestion with restriction endonucleases NheI and AscI.

The present disclosure also provides a method for targeted silencing of a plurality of genes, including: transforming the vector described above or the tandem vector described above into a target cell to obtain a multi-gene-silenced cell.

The present disclosure also provides multi-gene-silenced cells constructed by the method described above.

The present disclosure also provides a multi-gene-silenced homozygous cell line screened from the multi-gene-silenced cells described above.

Beneficial effects: The present disclosure provides a vector for multi-gene base editing. In the vector, a replicon element, U6, and sgRNA are linked in series to allow a plasmid carrying the replicon element (the epi element) to persist in a cell, such that other elements on the plasmid can be continuously expressed in the cell, which improves the efficiency of multi-gene silencing.

In the embodiments of the present disclosure, four genes associated with disease resistance (CALR, ANPEP, CD163, and ANTXR1) and a gene associated with a pork quality (MSTN) in the pig breeding field are selected for gene silencing. An epiBE4-5U6-sg vector is constructed by combining a replicon element with a sgRNA and a U6 promoter that are linked in series for each gene. The present disclosure also provides a tandem vector, which is obtained by removing the epi element from the vector above. In an embodiment, a BE4-5U6-sg vector is constructed. The two multi-gene editing vectors constructed in the embodiments of the present disclosure both have a silencing editing effect for target loci. The epiBE4-5U6-sg vector has a high editing efficiency. In the present disclosure, a homozygous cell line with 5 genes silenced is obtained through drug enrichment and screening. Thus, the present disclosure provides materials and methods for the disease-resistance breeding.

### BRIEF DESCRIPTION OF THE DRAWINGS

FIG. 1 is a plasmid map of pKLV2-U6gRNA (Bbs1)-PGKpuro-2AZsGreen;

FIG. 2 is a plasmid map of a vector epiBE4-5U6-sg;

FIG. 3 is a plasmid map of a vector BE4-5U6-sg;

FIGS. 4A-4E show cell editing efficiency results of the vector epiBE4-5U6-sg (abbreviated as epiBE4) and the vector BE4-5U6-sg (abbreviated as BE4) on day 3 (D3) and day 6 (D6) of enrichment;

FIGS. 5A-5C show images of single-cell assemblies on day 9 of cultivation with a high-concentration serum; and

FIG. 6 shows homozygous clones with 5 genes edited. FIG. 6 shows sequence fragments of gene ANPEP in WT (SEQ ID NO: 66) and sequence fragments of edited gene ANPEP in homozygous clones (SEQ ID NO:67); sequence fragments of gene MSTN in WT (SEQ ID NO: 68) and sequence fragments of edited gene MSTN in homozygous clones (SEQ ID NO: 69); sequence fragments of gene CD163 in WT (SEQ ID NO: 70) and sequence fragments of edited gene CD163 in homozygous clones (SEQ ID NO:71); sequence fragments of gene ANTXR1 in WT (SEQ ID NO: 72) and sequence fragments of edited gene ANTXR1 in homozygous clones (SEQ ID NO:73); sequence fragments of gene CALR in WT (SEQ ID NO: 74) and sequence fragments of edited gene CALR in homozygous clones (SEQ ID NO:75).

### DETAILED DESCRIPTION OF THE EMBODIMENTS

The present disclosure provides a vector for multi-gene base editing, including a replicon (epi) element, a U6 promoter, and sgRNA for each target gene that are linked in series. Each U6 promoter initiates expression of a sgRNA sequence of a target gene.

In a specific embodiment of the present disclosure, the epi element includes EBNA1 and OriP, where a sequence of the EBNA1 is shown in SEQ ID NO: 1 and a sequence of the OriP is shown in SEQ ID NO: 2.

In a specific embodiment of the present disclosure, a nucleotide sequence of the U6 promoter may be shown in SEQ ID NO: 3. In the present disclosure, each U6 promoter initiates expression of sgRNA of a target gene.

In the present disclosure, there is no less than 1 target gene. In a specific embodiment, sgRNAs for 5 target genes are designed simultaneously for gene silencing, such as four genes associated with disease resistance (CALR, ANPEP, CD163, and ANTXR1) and a gene associated with a pork quality (MSTN) in the pig breeding field that are selected in an embodiment, and the sgRNA sequences shown in Table 1 are designed.

TABLE 1
List of sgRNA sequences

| Gene | Sequence | SEQ ID No. |
|---|---|---|
| CALR sg 1 F | CACCGATCCCCGGAGTACCCTTATG | 4 |
| CALR sg 1 R | AAACCATAAGGGTACTCCGGGGATC | 5 |
| MSTN sg 1 F | CACCGCAGCGAGCAAAAGGAAAATG | 6 |
| MSTN sg 1 R | AAACCATTTTCCTTTTGCTCGCTGC | 7 |
| MSTN sg 3 F | CACCGAAACAACCTGAATCCAACTT | 8 |
| MSTN sg 3 R | AAACAAGTTGGATTCAGGTTGTTTC | 9 |
| MSTN sg 4 F | CACCGGTGCACCAAGCAAACCCCAG | 10 |
| MSTN sg 4 R | AAACCTGGGGTTTGCTTGGTGCACC | 11 |
| ANPEP sg 1 F | CACCGCCCCCAGTTCTCCATGGCAC | 12 |
| ANPEP sg 1 R | AAACGTGCCATGGAGAACTGGGGGC | 13 |
| ANPEP sg 2 F | CACCGGGATCGATGGACCCTGCAGA | 14 |
| ANPEP sg 2 R | AAACTCTGCAGGGTCCATCGATCCC | 15 |
| ANPEP sg 3 F | CACCGGCCCCCAGGCAAAGTCCCAC | 16 |
| ANPEP sg 3 R | AAACGTGGGACTTTGCCTGGGGGCC | 17 |
| ANTXR1 sg 1 F | CACCGGAACTCCAGAAGGTTCTGCC | 18 |
| ANTXR1 sg 1 R | AAACGGCAGAACCTTCTGGAGTTCC | 19 |
| ANTXR1 sg 2 F | CACCGTCCTTTCAAGTAGTGGTGAG | 20 |
| ANTXR1 sg 2 R | AAACCTCACCACTACTTGAAAGGAC | 21 |
| ANTXR1 sg 3 F | CACCGCTTCCGACACGCCCGCAATG | 22 |
| ANTXR1 sg 3 R | AAACCATTGCGGGCGTGTCGGAAGC | 23 |
| ANTXR1 sg 4 F | CCGACCAGAGGAGAGCCAGGGCC | 24 |
| ANTXR1 sg 4 R | AAACGGCCCTGGCTCTCCTCTGGTC | 25 |
| ANTXR1 sg 5 F | CACCGGAACCACCAGAGGAGAGCCA | 26 |
| ANTXR1 sg 5 R | AAACTGGCTCTCCTCTGGTGGTTCC | 27 |
| CD163 sg 1 F | CACCGTGGTCGAGTTAACGCCAGTG | 28 |
| CD163 sg 1 R | AAACCACTGGCGTTAACTCGACCAC | 29 |
| CD163 sg 2 F | CACCGGTCCCAGTGAGAGTTGCAGA | 30 |
| CD163 sg 2 R | AAACTCTGCAACTCTCACTGGGACC | 31 |
| CD163 sg 3 F | CACCGGTGTGCCGACAGCTGGGCTG | 32 |
| CD163 sg 3 R | AAACCAGCCCAGCTGTCGGCACACC | 33 |
| CD163 sg 4 F | CACCGAAGTACAACATGGAGACACG | 34 |
| CD163 sg 4 R | AAACCGTGTCTCCATGTTGTACTTC | 35 |

In the present disclosure, a backbone vector for the vector needs to include the following core elements: a single-base editor, an epi replicon element, and a plurality of U6 (or csy4) promoters linked in series. For example, in an embodiment, epiBE4-U6 is selected.

In the present disclosure, when the vector is constructed, for example, in an embodiment, the sgRNA is annealed and then linked with a U6 promoter to obtain a sg-U6 linked sequence for a gene, namely, U6-sg. The sg-U6 linked sequences for the five genes are linked in series, that is, U6-sg1, U6-sg2, U6-sg3, U6-sg4, and U6-sg5 are linked in series to obtain a sequence shown in SEQ ID NO: 36.

A universal vector epiBE4-U6 is selected as a sgRNA expression vector. A backbone of the vector is subjected to enzyme digestion with PaqCI, an enzyme digestion product is recovered, and the sequence shown in SEQ ID NO: 36 is ligated to the enzyme digestion product to construct a vector epiBE4-5U6-sg. A sequence of the epiBE4-U6 is shown in SEQ ID NO: 37.

The present disclosure also provides a tandem vector for multi-gene base editing, where a preparation method of the tandem vector includes: the replicon element is removed from the vector described above.

In a specific embodiment of the present disclosure, the preparation method of the tandem vector includes: the vector epiBE4-5U6-sg described above is subjected to double enzyme digestion with restriction endonucleases NheI and AscI, then linear ligation fragments of NheI and AscI are designed, and the above fragments are ligated to successfully construct a vector BE4-5U6-sg.

The present disclosure also provides a method for targeted silencing of a plurality of genes, including: the vector described above or the tandem vector described above is transformed into a target cell to obtain a multi-gene-silenced cell.

The present disclosure does not have a special limitation on a method of the transformation, and a conventional method in the art may be adopted for the transformation, such as electrotransformation.

The present disclosure also provides multi-gene-silenced cells constructed by the method described above.

In an embodiment of the present disclosure, recovered Landrace porcine fetal fibroblasts (PFFs) are electrotransformed with the vector or the tandem vector constructed above, and target fragments of edited genes are amplified through polymerase chain reaction (PCR). Results show that the above two multi-gene editing vectors both have a silencing editing effect for target loci.

The present disclosure also provides a multi-gene-silenced homozygous cell line screened from the multi-gene-silenced cells described above.

In an embodiment, the screening of the present disclosure includes: The multi-gene-silenced cells are cultivated with a medium including a serum at a high concentration (20%) for 9 d until cell assemblies are formed from single cells, during which the medium is changed every 2 d. Then cell clone assemblies are picked with a cloning cylinder and cultivated in a 48-well plate until the plate is fully covered with cells. ½ of the cells are collected and identified for a genotype. The remaining cells are transferred to a 24-well plate and cultivated. Monoclones identified as homozygous editing are cryopreserved with a cryopreservation solution and stored in a liquid nitrogen tank, which are the multi-gene-silenced homozygous cell line of the present disclosure.

In order to further illustrate the present disclosure, the vector for multi-gene base editing, the construction method, and the use provided by the present disclosure are described in detail below in connection with examples, but these examples should not be construed as limiting the claimed scope of the present disclosure.

Unless otherwise specified, the experimental methods described in the following examples are all conventional methods. The methods shall be conducted in accordance with the techniques or conditions described in the literature in the art or in accordance with the product specification. All materials and reagents used in the following examples may be commercially available, unless otherwise specified.

### Example 1

Amplification Primers were Designed for Target Loci of Five Genes, and sgRNA Expression Vectors were Constructed for Five Gene Loci.

### Verification of Genomic Single-Nucleotide Polymorphisms (SNPs)

A pair of primers were designed for an intron GGGG mutation of a CALR gene (CALR calreticulin [Sus scrofa (pig)] Gene ID: 100381266). Three pairs of primers were designed for exons 6, 11, and 18 of an ANPEP gene (ANPEP alanyl aminopeptidase, membrane [Sus scrofa (pig)] Gene ID: 397520). Three pairs of primers were designed for exons 1, 2, and 3 of an MSTN gene (MSTN myostatin [Sus scrofa (pig)] Gene ID: 399534). Three pairs of primers were designed for exons 4, 10, and 13 of an ANTXR1 gene (ANTXR1 ANTXR cell adhesion molecule 1 [Sus scrofa (pig)] Gene ID: 100513853). Four pairs of primers were designed for exons 5, 7, 8, and 12 of a CD163 gene (CD163 CD163 molecule [Sus scrofa (pig)] Gene ID: 397031). The designed upstream and downstream primers were used to amplify a genome of Yorkshire PFFs (a 35 d-pregnant Yorkshire sow was purchased from the Yangxiang Co., Ltd., piglet fibroblasts were isolated, and a genome of the fibroblasts was extracted) through PCC to obtain PCR products. Primer sequences were shown in Table 2.

TABLE 2
Primer sequences for PCR amplification

| Primer name | Primer sequence (5′-3′) | SEQ ID No. |
|---|---|---|
| ANPEP 1 F | ctccccatacccagagacca | 38 |
| ANPEP 1 R | cctcccttctctgtctgcac | 39 |
| ANPEP 2 F | cttccagcaaggcaccacta | 40 |
| ANPEP 2 R | cttggtgtccacggtgatga | 41 |
| ANPEP 3 F | ctgctgctccatccctgac | 42 |
| ANPEP 3 R | ctcacctgttcaggagccag | 43 |
| ANTXR1 1 F | gaatttcagggaacagatccgc | 44 |
| ANTXR1 1 R | ttgtgtacatgcttatgggaca | 45 |
| ANTXR1 2-3 F | tgcagtcccttaagctgtctt | 46 |
| ANTXR1 2-3 R | tgtatgaccttgccccgaag | 47 |
| ANTXR1 4-5 F | gtgttgcctcatccctctcc | 48 |
| ANTXR1 4-5 R | tttcagccacccaaccagag | 49 |
| CD163 1 F | ctgagagtggtagatggagtcac | 50 |
| CD163 1 R | gagcagactcgtgtccatggc | 51 |
| CD163 2 F | acaaatccgcttggtgaatgg | 52 |
| CD163 2 R | cggaacaatctcccatgtgct | 53 |
| CD163 3 F | acgtgtggagatctggtacg | 54 |
| CD163 3 R | ctggcaggacaatcccacaa | 55 |
| MSTN 1 F | atgctgattgttgctggtcc | 56 |
| MSTN 1 R | atcaatcagttcccggagtgg | 57 |
| MSTN 2-3 F | cgtcaagactcctacaacagtg | 58 |
| MSTN 2-3 R | cctggtcctgggaaggttaca | 59 |
| MSTN 4 F | atctcgatgctgtcgttaccc | 60 |
| MSTN 4 R | ggagacatctttgtgggagtaca | 61 |
| CALR 1 F | tccggcaaattctacggtga | 62 |
| CALR 1 R | agccctagcaagggacgata | 63 |

Amplification products were sent to a company for sequencing, and sequencing results were aligned with the corresponding sequences of the National Center for Biotechnology Information (NCBI). Alignment results show that the amplification products were normal and did not have SNPs.

### Construction of Expression Vectors

1. In order to construct efficient vectors, sgRNAs with high efficiencies and low off-target rates were designed for specific genes with the crisprbets website. A gRNA (CALR-sg1) was designed for the intron GGGG mutation of the CALR gene. Three gRNAs (ANPEP-sg1, ANPEP-sg2, and ANPEP-sg3) were designed for the exons 6, 11, and 18 of the ANPEP gene. Four gRNAs (MSTN-sg1, MSTN-sg2, MSTN-sg3, and MSTN-sg4) were designed for the exons 1, 2, and 3 of the MSTN gene. Five gRNAs (ANTXR1-sg1, ANTXR1-sg2, ANTXR1-sg3, ANTXR1-sg4, and ANTXR1-sg5) were designed for the exons 4, 10, and 13 of the ANTXR1 gene. Four gRNAs (CD163-sg1, CD163-sg2, CD163-sg3, and CD163-sg4) were designed for the exons 5, 7, 8, and 12 of the CD163 gene. Annealing primers for these gRNAs were designed and synthesized correspondingly, as shown in Table 1.

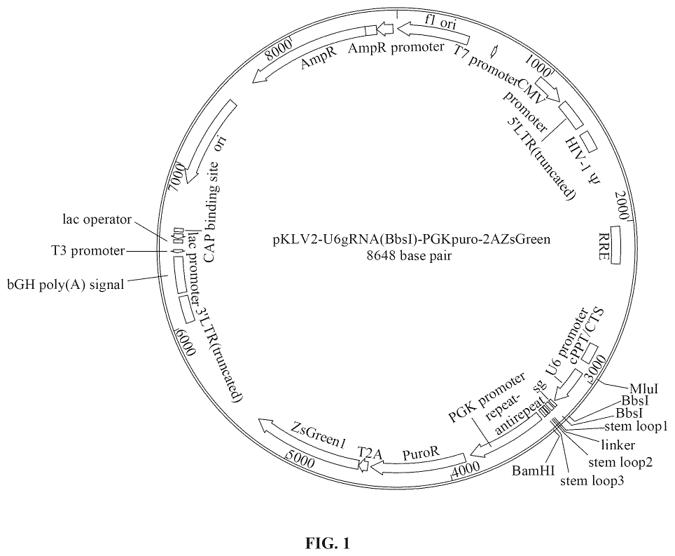

2. The pKLV2-U6gRNA (Bbs1)-PGKpuro-2AZsGreen shown in FIG. 1 was selected as a sgRNA expression vector. A backbone of the vector was subjected to enzyme digestion with Bbs1. A 50 μL enzyme digestion system was prepared with 3 μg of the pKLV2-U6gRNA (Bbs1)-PGKpuro-2AZsGreen plasmid, 5 μL of 10×rCutSmart Buffer, 1 μL of Bbs1-HF, and the balance of H₂O. A product generated after 3 h of the enzyme digestion was recovered to obtain an enzyme digestion product.

3.5 μL of each sgRNA oligonucleotide sequence with complementarily-paired sticky ends in Table 1 was taken and added to a PCR tube, shaken for thorough mixing, and then annealed in a PCR instrument to obtain an annealing product. An annealing procedure was as follows: 95° C. for 10 min, and 65° C. for 30 min.

4. The annealing product was ligated to the enzyme digestion product with a DNA ligase. A 10 μL ligation system was prepared with 50 ng of the enzyme digestion product pKLV2-U6gRNA (Bbs1)-PGKpuro-2AZsGreen, 1 μL of the annealing product, 5 μL of DNA ligation mix, and the balance of H₂O.

The ligation system was thoroughly mixed and then incubated in a constant-temperature metal bath at 25° C. for 10 min. After the ligation was completed, a ligation product was taken out and added to DH5a competent cells to obtain a mixture. The mixture was incubated in an ice bath for 5 min, then in a 42° C. water bath for 45 s, and then in an ice bath for 2 min, and then was coated on a solid medium for transformation. The solid medium was inverted and incubated overnight, monoclonal colonies with an appropriate size were picked and added to a 1.5 mL centrifuge tube, and 700 μL of an LB liquid medium was added to the centrifuge tube. The monoclonal colonies were cultivated on a shaker at 37° C. and 220 r/min for 8 h, and then sent to a company for sequencing. When it was confirmed that a vector was successfully constructed, the expanded cultivation was conducted, and the plasmid was extracted.

### Example 2

Cell Experiments were Conducted to Verify the Editing Efficiencies of Different sgRNAs for Each Gene.

A PK15 BE4 cell line was inoculated in a 10 cm dish and cultivated until a cell density was 90% to 100%, and then the sg plasmid vector was electrotransformed. The plasmid was transfected with 10 μg of the plasmid per ⅓ of the 10 cm dish. 72 h after the transfection, the flow cytometry sorting was conducted to sort green fluorescent protein (GFP)-positive cells out. A genome of the GFP-positive cells was extracted and amplified through PCR with the previous genome amplification primers. Each amplification product was sent to a company for sequencing, and an editing efficiency of each sgRNA was analyzed with the online website http://baseeditr.com/. sgRNA with the optimal activity for each gene locus was finally selected for the subsequent multi-gene editing experiment.

The sgRNAs finally selected were CALR sg 1, MSTN sg 4, ANPEP sg 1, ANTXR1 sg 2, and CD163 sg 4 shown in Table 1.

### Example 3

### Construction of Four Tandem Five-Gene-Knockout (KO) Vectors

1. Construction of a Vector epiBE4-5U6-Sg

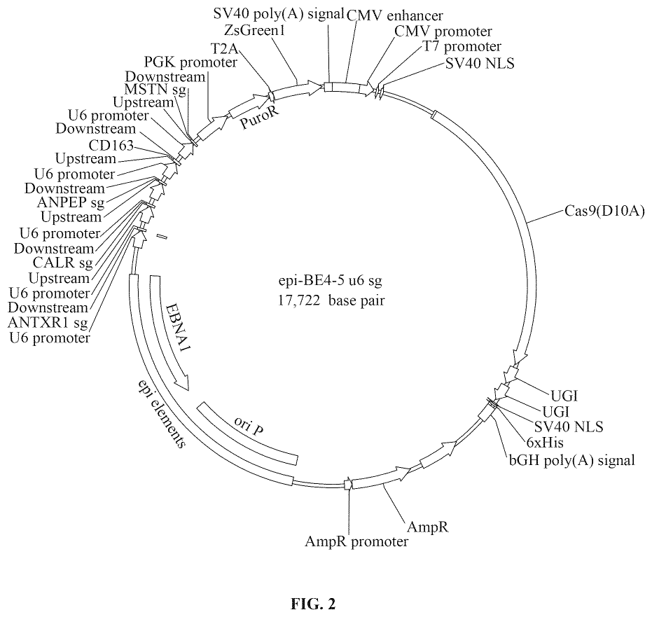

Each pair of U6 promoter+sgRNA for silencing of 5 genes was combined with the epi element to construct the vector epiBE4-5U6-sg. The sgRNAs of five genes were linked in series by linking sgRNA for each gene to a U6 promoter. That is, each U6 promoter initiated one sgRNA. A sequence shown in SEQ ID NO: 36 was finally obtained and cloned into a universal vector epiBE4-U6 to finally synthesize the vector epiBE4-5U6-sg shown in FIG. 2.

### 2. Construction of a Tandem Vector BE4-5U6-Sg

(1) The vector was subjected to double enzyme digestion. That is, on the basis of the vector epiBE4-5U6-sg, the EBNA1 and oriP elements were enzymatically removed. The double enzyme digestion was conducted with restriction endonucleases NheI and AscI.

A 50 μL enzyme digestion system was prepared with 1 μg of the plasmid, 5 μL of 10×rCutSmart Buffer, 1 μL of NheI-HF, 1 μL of AscI-HF, and the balance of H₂O, where

NheI F (SEQ ID NO: 64):
CTAGCAATTACTCGCAGCCCGGAA;
and
AscI R (SEQ ID NO: 65):
CGCGTTCCGGGCTGCGAGTAATTG.

(2) Two Linear Ligation Fragments were Designed to Ligate a Vector Undergoing Double Enzyme Digestion.

The two linear ligation fragments each of 5 μL were taken and thoroughly mixed under shaking, and then annealed in a PCR instrument to obtain an annealing product. An annealing procedure was as follows: 95° C. for 10 min, and 65° C. for 30 min. The annealing product was diluted with 90 μL of sterilized water.

### (3) Ligation

The annealing product and the linearized vector undergoing double enzyme digestion were ligated with a DNA Ligation Kit. A 10 μL ligation system was prepared with 100 ng of the linearized vector undergoing double enzyme digestion, 1 μL of the annealing product, 5 μL of DNA ligation mix, and the balance of H₂O. The ligation was conducted at 25° C. for 10 min to obtain a ligation product. Then the ligation product was transformed. Positive clones were identified through sequencing, endotoxins were removed, and the plasmid was extracted for later use. According to the above steps, the vector BE4-5U6-sg shown in FIG. 3 was successfully constructed.

### Example 4

Through Cell Experiments, it was Proved that the Multi-Gene Editing Vectors could Allow the Silencing of Five Genes in a Targeted Manner.
1. The Replicon Vector and the Conventional Vector Each were Electrotransformed into Landrace Fibroblasts, and the Cell Enrichment was Conducted with a Drug.

Landrace PFFs were recovered in a 10 cm dish, and when a cell density reached 100%, the electrotransformation was conducted. When the electrotransformation was conducted at a cell density of 100%, ⅓ of cells in the 10 cm dish were taken and electrotransformed with 10 μg of the plasmid epiBE4-5U6-sg at a voltage of 520 V, ⅓ of the cells in the 10 cm dish were taken and electrotransformed with 10 μg of the plasmid BE4-5U6-sg at a voltage of 520 V, and ⅓ of cells in the 10 cm dish were taken and electrotransformed directly without a plasmid at a voltage of 520 V as a negative control. After the electrotransformation was completed, each group of cells were transferred to an antibiotic-free medium. When it was found the next day that the cells had a normal morphology, the enrichment was conducted with puro (puro concentration: 1 μg/mL) for 3 d. Cells were taken at an amount ⅓ of a density to detect an editing efficiency. Then the enrichment was conducted with puro (puro concentration: 2 μg/mL) once again for 3 d. Cells were taken at an amount ⅓ of a density to detect an editing efficiency.

### 2. Verification of D3 and D6 Efficiencies of Multi-Gene Silencing Vector-Containing Cell Pools

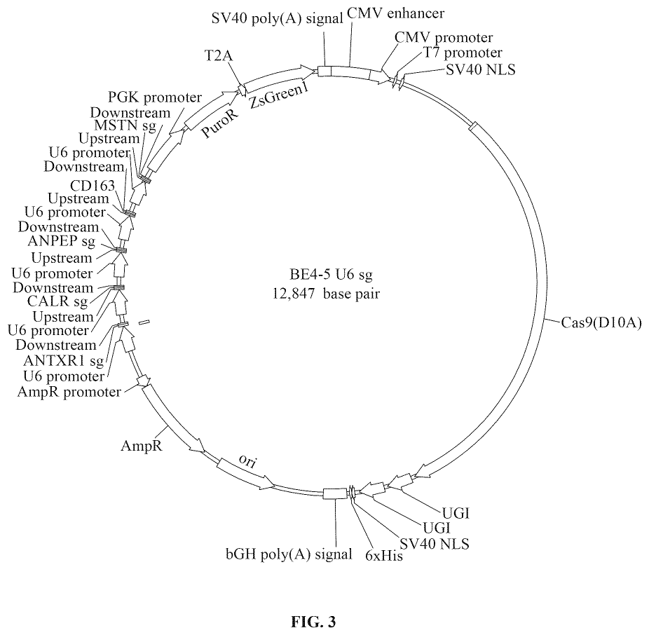

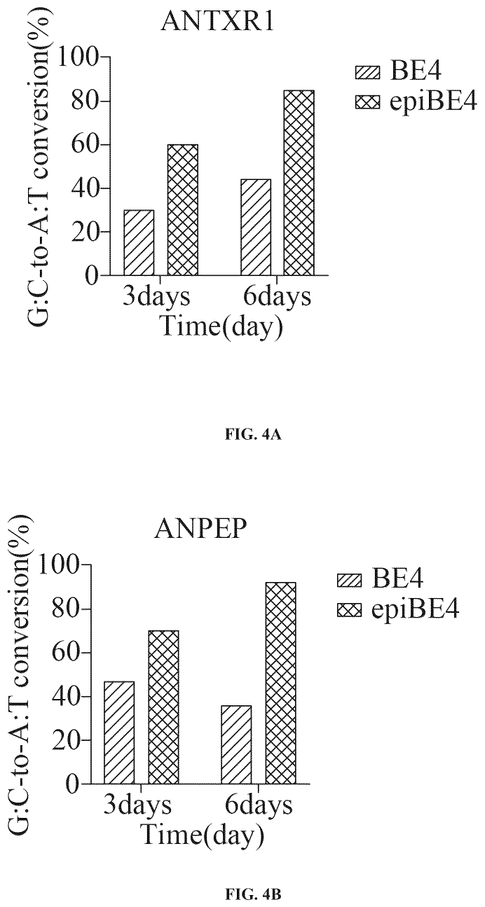

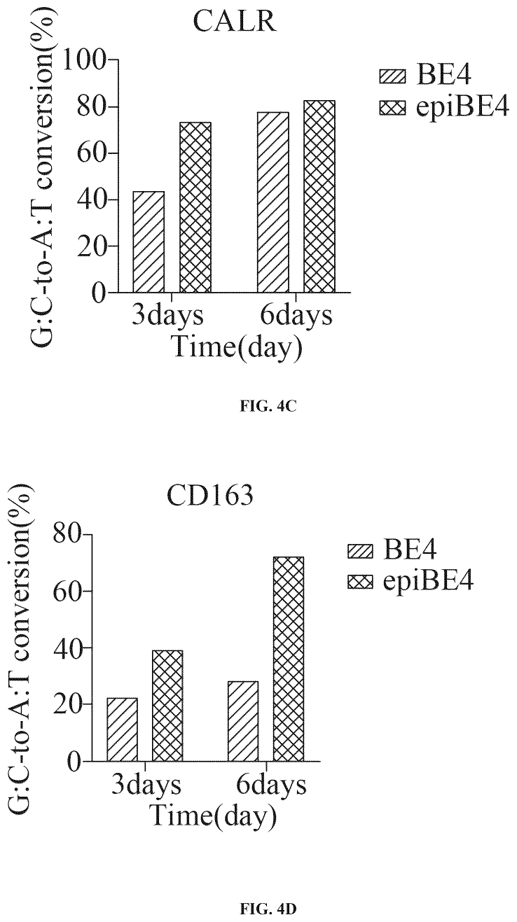

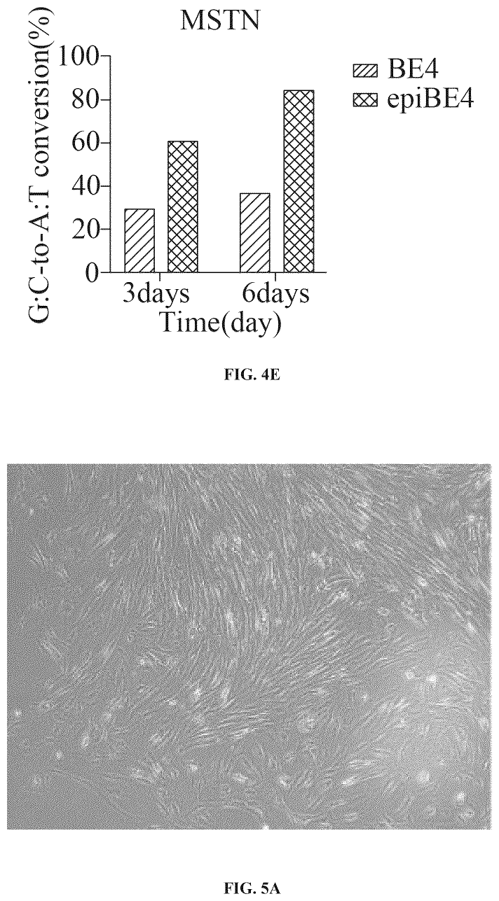

Target fragments of edited genes were amplified through PCR, and amplification products were sent to a company for testing. An editing efficiency of each gene locus was analyzed with the online website Edit® according to a peak chart of testing results. As shown in FIGS. 4A-4E, the two multi-gene editing vectors both have a silencing editing effect for target loci, and the replicon vector exhibits a maximum cell editing efficiency on D6 of enrichment.

### Example 5

Cell Experiments were Conducted to Prove that Homozygous Clones with 5 Genes Edited by the Vector epiBE4-5U6-Sg were Successfully Constructed.

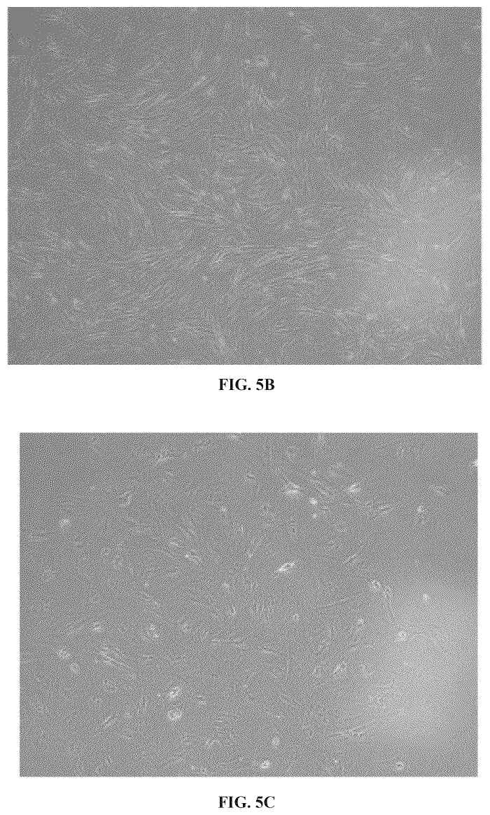

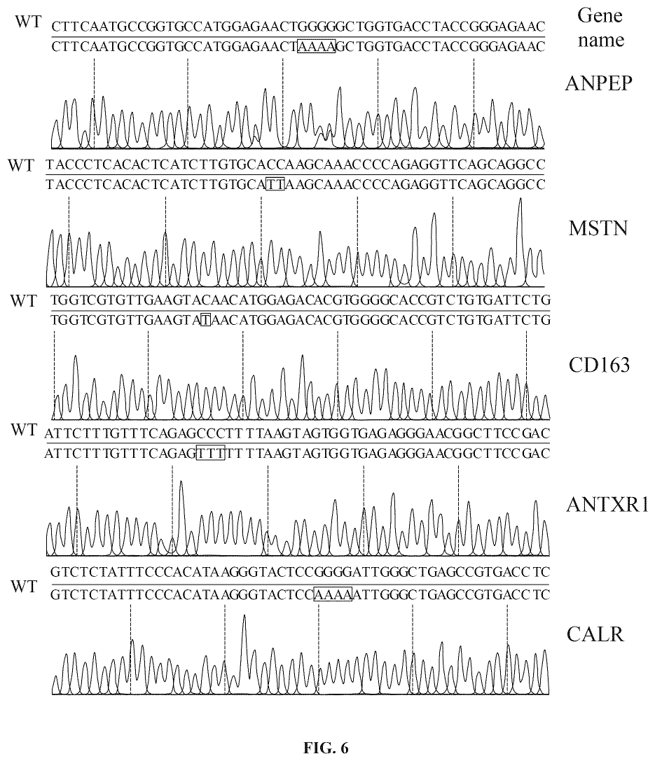

Landrace PFFs that were electrotransformed with epiBE4-5U6-sg and enriched for 6 d in Example 4 were subjected to serial dilution to obtain a 10 cm dish with 100 cells, and then the cells were cultivated with a medium including a high-concentration serum (20%, normal serum concentration: 10%), during which the medium was changed every 2 d. After 9 d of the cultivation, assemblies were formed from single cells, as shown in FIGS. 5A-5C. Cell clone assemblies were picked with a cloning cylinder and cultivated in a 48-well plate until the plate was fully covered with cells. ½ of the cells were collected and identified for a genotype. The remaining cells were transferred to a 24-well plate and cultivated. Monoclones identified as homozygous editing, as shown in FIG. 6, were cryopreserved with a cryopreservation solution and stored in a liquid nitrogen tank.

Although the present disclosure has been described in detail through the above examples, the examples are merely some rather than all of the examples of the present disclosure. All other examples obtained by a person based on these examples without creative efforts shall fall within the protection scope of the present disclosure.

What is claimed is:

1. A vector for multi-gene base editing, comprising a replicon element, a U6 promoter, and sgRNA for each target gene that are linked in series,
wherein each U6 promoter initiates expression of a sgRNA sequence of a target gene.

2. The vector according to claim 1, wherein the replicon element comprises EBNA1 and OriP.

3. The vector according to claim 1, wherein there is no less than 1 target gene.

4. The vector according to claim 1, wherein the target gene comprises a gene related to a pig breeding field and a gene related to a pork quality.

5. The vector according to claim 1, wherein a backbone vector for the vector comprises the following elements: a single-base editor, an epi replicon element, and a plurality of U6 promoters linked in series.

6. A tandem vector for multi-gene base editing, wherein a preparation method of the tandem vector comprises: removing the replicon element from the vector according to claim 1.

7. The tandem carrier according to claim 6, wherein the preparation method of the tandem vector comprises: subjecting the vector to double enzyme digestion with restriction endonucleases NheI and AscI.

8. A method for targeted silencing of a plurality of genes, comprising: transforming the vector according to claim 1 into a target cell to obtain a multi-gene-silenced cell.

9. Multi-gene-silenced cells constructed by the method according to claim 8.

10. A multi-gene-silenced homozygous cell line screened from the multi-gene-silenced cells according to claim 8.

11. The tandem carrier according to claim 6, wherein the replicon element comprises EBNA1 and OriP.

12. The tandem carrier according to claim 6, wherein there is no less than 1 target gene.

13. The tandem carrier according to claim 6, wherein the target gene comprises a gene related to a pig breeding field and a gene related to a pork quality.

14. The tandem carrier according to claim 6, wherein a backbone vector for the vector comprises the following elements: a single-base editor, an epi replicon element, and a plurality of U6 promoters linked in series.

15. The method according to claim 8, wherein the replicon element comprises EBNA1 and OriP.

16. The method according to claim 8, wherein there is no less than 1 target gene.

17. The method according to claim 8, wherein the target gene comprises a gene related to a pig breeding field and a gene related to a pork quality.

18. The method according to claim 8, wherein a backbone vector for the vector comprises the following elements: a single-base editor, an epi replicon element, and a plurality of U6 promoters linked in series.

19. A method for targeted silencing of a plurality of genes, comprising: transforming the vector according to claim 6 into a target cell to obtain a multi-gene-silenced cell.

20. The method according to claim 19, wherein the preparation method of the tandem vector comprises: subjecting the vector to double enzyme digestion with restriction endonucleases NheI and AscI.


SEQUENCE LISTING (75 sequences)

SEQ ID NO: 1 - DNA, 1926 bp, synthetic construct
  source 1..1926: mol_type = other DNA; note = Sequence of the EBNA1
atgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtga

SEQ ID NO: 2 - DNA, 1790 bp, synthetic construct
  source 1..1790: mol_type = other DNA; note = Sequence of the OriP
aacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctg

SEQ ID NO: 3 - DNA, 241 bp, synthetic construct
  source 1..241: mol_type = other DNA; note = Nucleotide sequence of the U6 promoter
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 4 - DNA, 25 bp, synthetic construct
  source 1..25: mol_type = other DNA; note = sgRNA sequence of CALR sg 1 F
caccgatccccggagtacccttatg

SEQ ID NO: 5 - DNA, 25 bp, synthetic construct
  source 1..25: mol_type = other DNA; note = sgRNA sequence of CALR sg 1 R
aaaccataagggtactccggggatc

SEQ ID NO: 6 - DNA, 25 bp, synthetic construct
  source 1..25: mol_type = other DNA; note = sgRNA sequence of MSTN sg 1 F
caccgcagcgagcaaaaggaaaatg

SEQ ID NO: 7 - DNA, 25 bp, synthetic construct
  source 1..25: mol_type = other DNA; note = sgRNA sequence of MSTN sg 1 R
aaaccattttccttttgctcgctgc

SEQ ID NO: 8 - DNA, 25 bp, synthetic construct
  source 1..25: mol_type = other DNA; note = sgRNA sequence of MSTN sg 3 F
caccgaaacaacctgaatccaactt

SEQ ID NO: 9 - DNA, 25 bp, synthetic construct
  source 1..25: mol_type = other DNA; note = sgRNA sequence of MSTN sg 3 R
aaacaagttggattcaggttgtttc

SEQ ID NO: 10 - DNA, 25 bp, synthetic construct
  source 1..25: mol_type = other DNA; note = sgRNA sequence of MSTN sg 4 F
caccggtgcaccaagcaaaccccag

SEQ ID NO: 11 - DNA, 25 bp, synthetic construct
  source 1..25: mol_type = other DNA; note = sgRNA sequence of MSTN sg 4 R
aaacctggggtttgcttggtgcacc

SEQ ID NO: 12 - DNA, 25 bp, synthetic construct
  source 1..25: mol_type = other DNA; note = sgRNA sequence of ANPEP sg 1 F
caccgcccccagttctccatggcac

SEQ ID NO: 13 - DNA, 25 bp, synthetic construct
  source 1..25: mol_type = other DNA; note = sgRNA sequence of ANPEP sg 1 R
aaacgtgccatggagaactgggggc

SEQ ID NO: 14 - DNA, 25 bp, synthetic construct
  source 1..25: mol_type = other DNA; note = sgRNA sequence of ANPEP sg 2 F
caccgggatcgatggaccctgcaga

SEQ ID NO: 15 - DNA, 25 bp, synthetic construct
  source 1..25: mol_type = other DNA; note = sgRNA sequence of ANPEP sg 2 R
aaactctgcagggtccatcgatccc

SEQ ID NO: 16 - DNA, 25 bp, synthetic construct
  source 1..25: mol_type = other DNA; note = sgRNA sequence of ANPEP sg 3 F
caccggcccccaggcaaagtcccac

SEQ ID NO: 17 - DNA, 25 bp, synthetic construct
  source 1..25: mol_type = other DNA; note = sgRNA sequence of ANPEP sg 3 R
aaacgtgggactttgcctgggggcc

SEQ ID NO: 18 - DNA, 25 bp, synthetic construct
  source 1..25: mol_type = other DNA; note = sgRNA sequence of ANTXR1 sg 1 F
caccggaactccagaaggttctgcc

SEQ ID NO: 19 - DNA, 25 bp, synthetic construct
  source 1..25: mol_type = other DNA; note = sgRNA sequence of ANTXR1 sg 1 R
aaacggcagaaccttctggagttcc

SEQ ID NO: 20 - DNA, 25 bp, synthetic construct
  source 1..25: mol_type = other DNA; note = sgRNA sequence of ANTXR1 sg 2 F
caccgtcctttcaagtagtggtgag

SEQ ID NO: 21 - DNA, 25 bp, synthetic construct
  source 1..25: mol_type = other DNA; note = sgRNA sequence of ANTXR1 sg 2 R
aaacctcaccactacttgaaaggac

SEQ ID NO: 22 - DNA, 25 bp, synthetic construct
  source 1..25: mol_type = other DNA; note = sgRNA sequence of ANTXR1 sg 3 F
caccgcttccgacacgcccgcaatg

SEQ ID NO: 23 - DNA, 25 bp, synthetic construct
  source 1..25: mol_type = other DNA; note = sgRNA sequence of ANTXR1 sg 3 R
aaaccattgcgggcgtgtcggaagc

SEQ ID NO: 24 - DNA, 23 bp, synthetic construct
  source 1..23: mol_type = other DNA; note = sgRNA sequence of ANTXR1 sg 4 F
ccgaccagaggagagccagggcc

SEQ ID NO: 25 - DNA, 25 bp, synthetic construct
  source 1..25: mol_type = other DNA; note = sgRNA sequence of ANTXR1 sg 4 R
aaacggccctggctctcctctggtc

SEQ ID NO: 26 - DNA, 25 bp, synthetic construct
  source 1..25: mol_type = other DNA; note = sgRNA sequence of ANTXR1 sg 5 F
caccggaaccaccagaggagagcca

SEQ ID NO: 27 - DNA, 25 bp, synthetic construct
  source 1..25: mol_type = other DNA; note = sgRNA sequence of ANTXR1 sg 5 R
aaactggctctcctctggtggttcc

SEQ ID NO: 28 - DNA, 25 bp, synthetic construct
  source 1..25: mol_type = other DNA; note = sgRNA sequence of CD163 sg 1 F
caccgtggtcgagttaacgccagtg

SEQ ID NO: 29 - DNA, 25 bp, synthetic construct
  source 1..25: mol_type = other DNA; note = sgRNA sequence of CD163 sg 1 R
aaaccactggcgttaactcgaccac

SEQ ID NO: 30 - DNA, 25 bp, synthetic construct
  source 1..25: mol_type = other DNA; note = sgRNA sequence of CD163 sg 2 F
caccggtcccagtgagagttgcaga

SEQ ID NO: 31 - DNA, 25 bp, synthetic construct
  source 1..25: mol_type = other DNA; note = sgRNA sequence of CD163 sg 2 R
aaactctgcaactctcactgggacc

SEQ ID NO: 32 - DNA, 25 bp, synthetic construct
  source 1..25: mol_type = other DNA; note = sgRNA sequence of CD163 sg 3 F
caccggtgtgccgacagctgggctg

SEQ ID NO: 33 - DNA, 25 bp, synthetic construct
  source 1..25: mol_type = other DNA; note = sgRNA sequence of CD163 sg 3 R
aaaccagcccagctgtcggcacacc

SEQ ID NO: 34 - DNA, 25 bp, synthetic construct
  source 1..25: mol_type = other DNA; note = sgRNA sequence of CD163 sg 4 F
caccgaagtacaacatggagacacg

SEQ ID NO: 35 - DNA, 25 bp, synthetic construct
  source 1..25: mol_type = other DNA; note = sgRNA sequence of CD163 sg 4 R
aaaccgtgtctccatgttgtacttc

SEQ ID NO: 36 - DNA, 1816 bp, synthetic construct
  source 1..1816: mol_type = other DNA; note = Sequence of sg-U6 linked sequences for the five genes linked in series
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcctttcaagtagtggtgaggtttaagagctatgctggaaacagcatagcaagtttaaataaggctagtccgttatcaacttgaaaaagtggcaccgagtcggtgctttttttgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatccccggagtacccttatggtttaagagctatgctggaaacagcatagcaagtttaaataaggctagtccgttatcaacttgaaaaagtggcaccgagtcggtgctttttttgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcccccagttctccatggcacgtttaagagctatgctggaaacagcatagcaagtttaaataaggctagtccgttatcaacttgaaaaagtggcaccgagtcggtgctttttttgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaagtacaacatggagacacggtttaagagctatgctggaaacagcatagcaagtttaaataaggctagtccgttatcaacttgaaaaagtggcaccgagtcggtgctttttttgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgcaccaagcaaaccccaggtttaagagctatgctggaaacagcatagcaagtttaaataaggctagtccgttatcaacttgaaaaagtggcaccgagtcggtgctttttttt

SEQ ID NO: 37 - DNA, 16988 bp, synthetic construct
  source 1..16988: mol_type = other DNA; note = Sequence of the epiBE4-U6
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggcctaatacgactcactatagggagagccgccaccatgaaacggacagccgacggaagcgagttcgagtcaccaaagaagaagcggaaagtcagcagtgaaaccggaccagtggcagtggacccaaccctgaggagacggattgagccccatgaatttgaagtgttctttgacccaagggagctgaggaaggagacatgcctgctgtacgagatcaagtggggcacaagccacaagatctggcgccacagctccaagaacaccacaaagcacgtggaagtgaatttcatcgagaagtttacctccgagcggcacttctgcccctctaccagctgttccatcacatggtttctgtcttggagcccttgcggcgagtgttccaaggccatcaccgagttcctgtctcagcaccctaacgtgaccctggtcatctacgtggcccggctgtatcaccacatggaccagcagaacaggcagggcctgcgcgatctggtgaattctggcgtgaccatccagatcatgacagccccagagtacgactattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcaccattgctctgcagtcttgtcactatcagcggctgcctcctcatattctgtgggctacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctaaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtctactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcggctagcgttggcgggtgtcggggcgcagccatgaggtcgactctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataagccccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcggcgcgccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccatgcgcaggtggtagtcaaaagcctccggtcggaggcttttgacttggctgaggagtgccattaagcaccggtggagtgacgaccttcagcacgttcgtactgttcaacgatggtgtagtcttcgttgtgggaggtgatgtccagtttgatgtcggttttgtaagcacccggcagctgaaccggttttttagccatgtaggtggttttaacttcagcgtcgtagtgaccaccgtctttcagtttcagacgcattttgatttcacctttcagagcaccgtcttccgggtacatacgttcggtggaagcttcccaacccatggtttttttctgcataaccggaccgtcggacgggaagttggtaccacgcagtttaactttgtagatgaactcaccgtcttgcagggaggagtcctgggtaacggtaacaacaccaccgtcttcgaagttcataacacgttcccatttgaaaccttccgggaaggacagtttcaggtagtccgggatgtcagccgggtgtttaacgtaagctttggaaccgtactggaactgcggggacaggatgtcccaagcgaacggcagcggaccacctttggtaactttcagtttagcggtctgggtaccttcgtacggacgaccttcaccttcaccttcgatttcgaactcgtgaccgttaacggaaccttccatacgaactttgaaacgcatgaactctttgataacgtcttcgctactcgccatagatcctttctcctctttcagatccgtgctcagtatctctatcactgatagggatgtcaatctctatcactgatagggaggtctcgttttttttgcaggtggggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatccatctagatctcgagcagctgaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggcggcgggtccggaggagagggcagaggaagtctcctaacatgcggtgacgtggaggagaatcctggcccagaattcatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctaacccgatgtacgggccagatatacgcgttaacttgtttattgcagcttataatggttacaaataaagcaatagcatcacaaatttcacaaataaagcatttttttcactgcattctagttgtggtttgtccaaactcatcaatgtatctta

SEQ ID NO: 38 - DNA, 20 bp, synthetic construct
  source 1..20: mol_type = other DNA; note = Primer ANPEP 1 F
ctccccatacccagagacca

SEQ ID NO: 39 - DNA, 20 bp, synthetic construct
  source 1..20: mol_type = other DNA; note = Primer ANPEP 1 R
cctcccttctctgtctgcac

SEQ ID NO: 40 - DNA, 20 bp, synthetic construct
  source 1..20: mol_type = other DNA; note = Primer ANPEP 2 F
cttccagcaaggcaccacta

SEQ ID NO: 41 - DNA, 20 bp, synthetic construct
  source 1..20: mol_type = other DNA; note = Primer ANPEP 2 R
cttggtgtccacggtgatga

SEQ ID NO: 42 - DNA, 19 bp, synthetic construct
  source 1..19: mol_type = other DNA; note = Primer ANPEP 3 F
ctgctgctccatccctgac

SEQ ID NO: 43 - DNA, 20 bp, synthetic construct
  source 1..20: mol_type = other DNA; note = Primer ANPEP 3 R
ctcacctgttcaggagccag

SEQ ID NO: 44 - DNA, 22 bp, synthetic construct
  source 1..22: mol_type = other DNA; note = Primer ANTXR1 1 F
gaatttcagggaacagatccgc

SEQ ID NO: 45 - DNA, 22 bp, synthetic construct
  source 1..22: mol_type = other DNA; note = Primer ANTXR1 1 R
ttgtgtacatgcttatgggaca

SEQ ID NO: 46 - DNA, 21 bp, synthetic construct
  source 1..21: mol_type = other DNA; note = Primer ANTXR1 2-3 F
tgcagtcccttaagctgtctt

SEQ ID NO: 47 - DNA, 20 bp, synthetic construct
  source 1..20: mol_type = other DNA; note = Primer ANTXR1 2-3 R
tgtatgaccttgccccgaag

SEQ ID NO: 48 - DNA, 20 bp, synthetic construct
  source 1..20: mol_type = other DNA; note = Primer ANTXR1 4-5 F
gtgttgcctcatccctctcc

SEQ ID NO: 49 - DNA, 20 bp, synthetic construct
  source 1..20: mol_type = other DNA; note = Primer ANTXR1 4-5 R
tttcagccacccaaccagag

SEQ ID NO: 50 - DNA, 23 bp, synthetic construct
  source 1..23: mol_type = other DNA; note = Primer CD163 1 F
ctgagagtggtagatggagtcac

SEQ ID NO: 51 - DNA, 21 bp, synthetic construct
  source 1..21: mol_type = other DNA; note = Primer CD163 1 R
gagcagactcgtgtccatggc

SEQ ID NO: 52 - DNA, 21 bp, synthetic construct
  source 1..21: mol_type = other DNA; note = Primer CD163 2 F
acaaatccgcttggtgaatgg

SEQ ID NO: 53 - DNA, 21 bp, synthetic construct
  source 1..21: mol_type = other DNA; note = Primer CD163 2 R
cggaacaatctcccatgtgct

SEQ ID NO: 54 - DNA, 20 bp, synthetic construct
  source 1..20: mol_type = other DNA; note = Primer CD163 3 F
acgtgtggagatctggtacg

SEQ ID NO: 55 - DNA, 20 bp, synthetic construct
  source 1..20: mol_type = other DNA; note = Primer CD163 3 R
ctggcaggacaatcccacaa

SEQ ID NO: 56 - DNA, 20 bp, synthetic construct
  source 1..20: mol_type = other DNA; note = Primer MSTN 1 F
atgctgattgttgctggtcc

SEQ ID NO: 57 - DNA, 21 bp, synthetic construct
  source 1..21: mol_type = other DNA; note = Primer MSTN 1 R
atcaatcagttcccggagtgg

SEQ ID NO: 58 - DNA, 22 bp, synthetic construct
  source 1..22: mol_type = other DNA; note = Primer MSTN 2-3 F
cgtcaagactcctacaacagtg

SEQ ID NO: 59 - DNA, 21 bp, synthetic construct
  source 1..21: mol_type = other DNA; note = Primer MSTN 2-3 R
cctggtcctgggaaggttaca

SEQ ID NO: 60 - DNA, 21 bp, synthetic construct
  source 1..21: mol_type = other DNA; note = Primer MSTN 4 F
atctcgatgctgtcgttaccc

SEQ ID NO: 61 - DNA, 23 bp, synthetic construct
  source 1..23: mol_type = other DNA; note = Primer MSTN 4 R
ggagacatctttgtgggagtaca

SEQ ID NO: 62 - DNA, 20 bp, synthetic construct
  source 1..20: mol_type = other DNA; note = Primer CALR 1 F
tccggcaaattctacggtga

SEQ ID NO: 63 - DNA, 20 bp, synthetic construct
  source 1..20: mol_type = other DNA; note = Primer CALR 1 R
agccctagcaagggacgata

SEQ ID NO: 64 - DNA, 24 bp, synthetic construct
  source 1..24: mol_type = other DNA; note = NheI F
ctagcaattactcgcagcccggaa

SEQ ID NO: 65 - DNA, 24 bp, synthetic construct
  source 1..24: mol_type = other DNA; note = AscI R
cgcgttccgggctgcgagtaattg

SEQ ID NO: 66 - DNA, 52 bp, synthetic construct
  source 1..52: mol_type = other DNA; note = Sequence fragments of gene ANPEP in WT
cttcaatgccggtgccatggagaactgggggctggtgacctaccgggagaac

SEQ ID NO: 67 - DNA, 52 bp, synthetic construct
  source 1..52: mol_type = other DNA; note = Sequence fragments of edited gene ANPEP in homozygous clones
cttcaatgccggtgccatggagaactaaaagctggtgacctaccgggagaac

SEQ ID NO: 68 - DNA, 52 bp, synthetic construct
  source 1..52: mol_type = other DNA; note = Sequence fragments of gene MSTN in WT
taccctcacactcatcttgtgcaccaagcaaaccccagaggttcagcaggcc

SEQ ID NO: 69 - DNA, 52 bp, synthetic construct
  source 1..52: mol_type = other DNA; note = Sequence fragments of edited gene MSTN in homozygous clones
taccctcacactcatcttgtgcattaagcaaaccccagaggttcagcaggcc

SEQ ID NO: 70 - DNA, 53 bp, synthetic construct
  source 1..53: mol_type = other DNA; note = Sequence fragments of gene CD163 in WT
tggtcgtgttgaagtacaatatggagacacgtggggcaccgtctgtgattctg

SEQ ID NO: 71 - DNA, 53 bp, synthetic construct
  source 1..53: mol_type = other DNA; note = Sequence fragments of edited gene CD163 in homozygous clones
tggtcgtgttgaagtataacatggagacacgtggggcaccgtctgtgattctg

SEQ ID NO: 72 - DNA, 53 bp, synthetic construct
  source 1..53: mol_type = other DNA; note = Sequence fragments of gene ANTXR1 in WT
attctttgtttcagagccctttaaagtagtggtgagagggaacggcttccgac

SEQ ID NO: 73 - DNA, 53 bp, synthetic construct
  source 1..53: mol_type = other DNA; note = Sequence fragments of edited gene ANTXR1 in homozygous clones
attctttgtttcagagtttttttaagtagtggtgagagggaacggcttccgac

SEQ ID NO: 74 - DNA, 53 bp, synthetic construct
  source 1..53: mol_type = other DNA; note = Sequence fragments of gene CALR in WT
gtctctatttcccacataagggtactccggggattgggctgagccgtgacctc

SEQ ID NO: 75 - DNA, 53 bp, synthetic construct
  source 1..53: mol_type = other DNA; note = Sequence fragments of edited gene CALR in homozygous clones
gtctctatttcccacataagggtactccaaaaattgggctgagccgtgacctc